(19) United States
(12) Patent Application Publication
(10) Pub. No.: US 2022/0339300 A1
(43) Pub. Date: Oct. 27, 2022

(54) HYBRID NANOPARTICLES COMPRISING MANGANESE OXIDE AND HIGHLY REDUCED GRAPHENE OXIDE FOR THERANOSTIC APPLICATIONS

(71) Applicant: King Saud University, Riyadh (SA)

(72) Inventors: Haseeb Ahmad Khan, Riyadh (SA); Mohammed Rafi Shaik, Riyadh (SA); Sara Tariq Alrashood, Riyadh (SA); Aishah Ali Ekhzaimy, Riyadh (SA)

(73) Assignee: King Saud University, Riyadh (SA)

(21) Appl. No.: 17/863,066

(22) Filed: Jul. 12, 2022

(51) Int. Cl. A61K 49/18; C01G 45/02; C01B 32/23; C01B 32/198; C01B 32/184; A61K 41/00; A61P 35/00

(52) U.S. Cl. CPC A61K 49/1818; C01G 45/02; C01B 32/23; C01B 32/198; C01B 32/184; A61K 41/0057; A61P 35/00; B82Y 5/00

(57) ABSTRACT

The present disclosure provides HRG-Mn₃O₄ hybrid nanoparticles. The HRG-Mn₃O₄ hybrid nanoparticles do not pose any cytotoxicity at normal physiological conditions and therefore they are nontoxic and biocompatible at physiological conditions. The HRG-Mn₃O₄ hybrid nanoparticles under exposure of laser light cause massive cellular damage indicating their potential use for photodynamic therapy of cancer. The HRG-Mn₃O₄ hybrid nanoparticles enhance the magnetic resonance signals from cancer cells and exhibit excellent MRI contrast property for tumor imaging and are therefore useful contrast agent.

### FIELD OF INVENTION

The embodiments of the present disclosure generally relate to hybrid nanoparticles. More particularly, the present disclosure relates to a hybrid nanoparticle comprising manganese oxide and highly reduced graphene oxide and uses thereof in theranostic applications for example photodynamic therapy of cancer and MRI imaging.

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

Chemotherapy and radiation therapies are very harsh and tiring treatments for cancer patients with side effects such as nausea, vomiting and hair loss. Chemotherapy medications are toxic because they cannot discriminate between cancer and normal cells so besides killing cancer cells, they also damage normal cells. Similarly, most of the contrast agents for MRI diagnosis of cancer are toxic. That is why there has been increasing focus on alternative options that can limit the side effects associated with contrast agents and chemotherapy of cancer.

Tumor ablation, a minimally invasive technique, is one such option, preferred for the treatment of different types of tumors such as liver, kidney, bone, and lungs. Not only it offers an alternative for failed chemotherapy or radiotherapy or for non-surgical candidates, but is considered as a first-line treatment in patients suffering from benign tumors in the liver or small hepatocellular carcinomas. Thermal ablation is carried out by heating or cooling the targeted tissue to cytotoxic levels. Tumor cells are generally more sensitive to heating than normal cells owing to variations in sensitivity to tissue hypoxia (Nikfarjam M, Muralidharan V, Christophi C., Mechanisms of focal heat destruction of liver tumors, J Surg Res. 2005: 208-223) and hydrogen ion concentration (Overgaard J., Influence of extracellular pH on the viability and morphology of tumor cells exposed to hyperthermia, J Natl Cancer Inst. 1976; 56: 1243-1250). Interstitial laser ablation is an additional hyperthermic ablation technique. Another technique of tumor ablation is, light generated by neodymium:yttrium aluminum garnet lasers (wavelength of 1064 nm), which is directed to the target tissue; the light is absorbed by the tissue and converted to heat for therapeutic purpose (Knavel E M, Brace C L., Tumor ablation: common modalities and general practices, Tech Vase Interv Radiol. 2013 16: 192-200).

Photodynamic therapy (PDT) is an alternate technique in which the cancer cells are exposed to light of specific wavelength after administration of nontoxic photosensitizers. The light-induced excitation of photosensitizers emits fluorescence and generates potentially toxic free radicals imparting photosensitizers the properties of imaging as well as therapeutic agent. One of the major disadvantages of PDT for combined imaging and treatment applications is the limited tissue penetration by visible light, for the activation of photosensitizers (Mac Donald I J, Dougherty T J., Basic principles of photodynamic therapy, J Porphyrin Phthalocyanin 2001; 5: 105-129).

Therefore, there remains a need to develop PDT agents that can be activated by light at of 620-750 nm which is called as ‘visible red optical window’ (Sekkat Nawal et al., Like a Bolt from the Blue: Phthalocyanines in Biomedical Optics, Molecules 2012, 17, 98-144). At these wavelengths, body tissues are transparent, and the visible red radiation can be used to activate photosensitizers in deep tumors without phototoxicity to normal tissue.

Graphene-based nanomaterials have been considered as one of the potential nanomedicine candidates. In contrast to other carbon-based materials, have been observed to present a larger surface area, are easier to functionalize and have improved solubility, due to their unique optical, physicochemical and biomedical properties which enhance their applications in nanomedicine. Graphene oxide (GRO) nanoparticles functionalized with other materials have shown promising theranostic properties for cancer diagnosis and therapy. However, effective use of GRO remains challenge as previous studies have shown that in a low concentration GRO has been observed not to enter cancer cells and are less toxic to different cell lines with a survival rate exceeding 80% at a high concentration of 200 μg/ml and the higher concentration of GRO has been found to cause oxidative stress and induced a slight loss of cell viability (Liao K H, Lin Y S, Christopher W M, Haynes C L, Cytotoxicity of graphene oxide and graphene in human erythrocytes and skin fibroblasts, ACS Appl Mater Interfaces 2011; 3: 2607-2615; and Chang J Y, Yang S T, Liu J H, Dong E, Wang Y, Cao A, Liu Y, Wang H., In vitro toxicity evaluation of graphene oxide on A549 cells, Toxicol Lett. 2011; 200: 201-210). Another study showed that graphene oxide is nontoxic at low and medium doses whereas high doses cause significant toxicity, both in-vitro and in-vivo, with a strong tendency to affect lung, liver, spleen and kidney (Wang K, Ruan J, Song H, Zhang J, Wo Y, Guo S, Cui D., Biocompatibility of graphene oxide, Nanoscale Res Lett. 2011; 6: 8).

Manganese oxides, viz., MnO, MnO₂, Mn₂O₃, and Mn₃O₄, are attractive candidates for novel MRI contrast agent due to their inherent properties based on electronic configuration that can produce a large magnetic moment and cause nearby water protons relaxation (Cai X, Zhu Q, Zeng Y, Zeng Q, Chen X, Zhau Y., Manganese oxide nanoparticles as MRI contrast agents in tumor multimodal imaging and therapy, International Journal of Nanomedicine 2019; 14: 8321-8344). Manganese oxide nanoparticles have been shown to be a promising T₁-weighted contrast agent with high magnetization spins and fast water proton exchange rates (Na H B, Lee J H, An K, Park Y, Park M, Lee I S, Nam D, Kim S T, Kim S H, Kim S W, Lim K H, Kim K S, Kim S O, Hyeon T., Development of a T1 contrast agent for magnetic resonance imaging using MnO nanoparticles, Angew, Chernie Int. Ed. 2007; 46: 5397-5401). Such properties therefore make manganese oxides as one of the most widely investigated nanomaterials for image-guided therapeutic purposes as well as MRI contrast agents for biomedical imaging and tumor diagnosis. Although, manganese oxide nanoparticles with good crystallinity can be synthesized on a large scale under mild and ambient reaction conditions, it is difficult to design and synthesize highly stable Mn²⁺ complexes with high sensitivities for clinical applications (Yu T, Moon J, Park J, Park Y I, Na 11B, Kim B I, Song I C, Moon W K, Hyeon T., Various-shaped uniform Mn₃O₄ nanocrystals synthesized at low temperature in air Atmosphere. Chem Mater. 2009; 21: 2272-2279).

Thus, there is an unmet need in the art to provide an agent can be effectively used as PDT agent to activate photosensitizers in tumors without phototoxicity to normal tissue for theranostic applications for example diagnosis and treatment of cancer.

### SUMMARY

This section is provided to introduce certain objects and aspects of the present invention in a simplified form that are further described below in the detailed description. This summary is not intended to identify the key features or the scope of the claimed subject matter.

In an aspect, the present disclosure discloses nanoparticles that are nontoxic and biocompatible at physiological conditions for theranostic applications.

In an aspect the present disclosure provides hybrid nanoparticles (HRG-Mn₃O₄) that are nontoxic and biocompatible at physiological conditions.

In an aspect the present disclosure provides a method of synthesis of hybrid nanoparticle comprising:
(i). synthesising manganese oxide (Mn₃O₄) nanoparticles;
(ii). synthesising highly reduced graphene oxide (HRG) nanoparticles; and
(iii). preparing highly reduced graphene-Mn₃O₄ (HRG-Mn₃O₄) hybrid nanoparticles comprising steps of milling (Mn₃O₄) nanoparticles and highly reduced graphene oxide (HRG) nanoparticles.

In an aspect the present disclosure provides use of (HRG-Mn₃O₄) hybrid nanoparticles for theranostic applications such as photodynamic therapy agent for treatment of cancer and/or as contrast agent for imaging of cancer cells.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry of each component. It will be appreciated by those skilled in the art that the invention of such drawings includes the invention of electrical components, electronic components, or circuitry commonly used to implement such components.

FIG. 1 illustrates characterization of HRG-Mn₃O₄ nanoparticles as per an exemplary embodiment of the present disclosure: (A) Transmission electron microscopy image, (B) Energy-dispersive X-ray spectra, (C) UV-visible spectroscopy spectra, (D) FT-IR spectra, (E) X-ray diffraction analysis spectra; and (F) thermogravimetric analysis graph.

FIG. 2 illustrates quantitative determination of hemolysis by HRG-Mn₃O₄ nanoparticles as per an exemplary embodiment of the present disclosure: (A) A graph depicting hemolysis (%) by measuring the absorbance of the supernatants at 540 nm of H₂O, PBS, and HRG-Mn₃O₄ nanoparticles at 100, 50, 25, and 12 μg/ml; and (B) digital image of tubes (inside) of said samples.

FIG. 3 illustrates cellular uptake of HRG-Mn₃O₄ nanoparticles with different concentrations exposure determined by ICP-MS.

FIG. 4 illustrates cytotoxicity analysis showing cell viability of A549 cells treated with different concentrations of Mn₃O₄ and HRG-Mn₃O₄ nanoparticles.

FIG. 5 illustrates cell viability of A549 cells incubated with different concentrations of PBS (control), Triton X100 (negative control), Mn₃O₄ and HRG-Mn₃O₄ nanoparticles in presence of 670 nm laser irradiation (0.1 W/cm²) for 5 min. Data are represented as mean±standard deviation (n=3). *P<0.05, **P<0.01 and ***P<0.001 versus respective control group.

FIG. 6 illustrates fluorescence microscopy images of A549 cells co-stained with fluorescein diacetate (green emission for live cells) and propidium iodide (red emission for dead cells) with PBS (control), HRG-Mn₃O₄ nanoparticles with/without laser irradiation (670 nm, 0.1 W/cm²) for 5 min.

FIG. 7 illustrates T₁-weighted MR imaging of HRG-Mn₃O₄ nanoparticles in aqueous solution and the T1 relaxivity plot of aqueous suspension of HRG-Mn₃O₄ nanoparticles.

FIG. 8 illustrates cellular T₁-weighted MR imaging of HRG-Mn₃O₄ nanoparticles. T₁-weighted MR images of HRG-Mn₃O₄ nanoparticles in A549 cells after 24 h incubation time.

The foregoing shall be more apparent from the following more detailed description of the invention.

### DETAILED DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the invention as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

Reference throughout this specification to “one embodiment” or “an embodiment” or “an instance” or “one instance” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The phrase “Theranostic application” means combined diagnostic and therapeutic applications by the same nanoparticles having dual functionality.

The present disclosure provides hybrid nanoparticles (HRG-Mn₃O₄) that are nontoxic and biocompatible at physiological conditions for theranostic applications for example diagnosis and treatment of cancer.

In an embodiment, the present disclosure provides (HRG-Mn₃O₄) hybrid nanoparticles useful in photodynamic therapy.

In an embodiment, the present disclosure provides (HRG-Mn₃O₄) hybrid nanoparticles useful as contrast agent to enhance the magnetic resonance signals from cancer cells.

In an embodiment, the present disclosure provides (HRG-Mn₃O₄) hybrid nanoparticles as photodynamic therapy and imaging agent for cancer.

In an embodiment, the hybrid nanoparticles (HRG-Mn₃O₄) in accordance of the present disclosure are round with the average diameter of about 8 nm to about 16 nm, preferably from about 9 nm to about 15 nm.

In an embodiment, the hybrid nanoparticles (HRG-Mn₃O₄) have the average diameter of about 8 nm, 9 nm, 10 nm, 11 nm, 12 nm, 13 nm, 14 nm, 15 nm, or 16 nm.

In an embodiment, the hybrid nanoparticle (HRG-Mn₃O₄) in accordance with the present disclosure exhibits X-ray photoelectron spectrum comprising the peaks at about 0.65 keV, 5.88 keV, and 6.62 keV, and 0.0-0.5 keV.

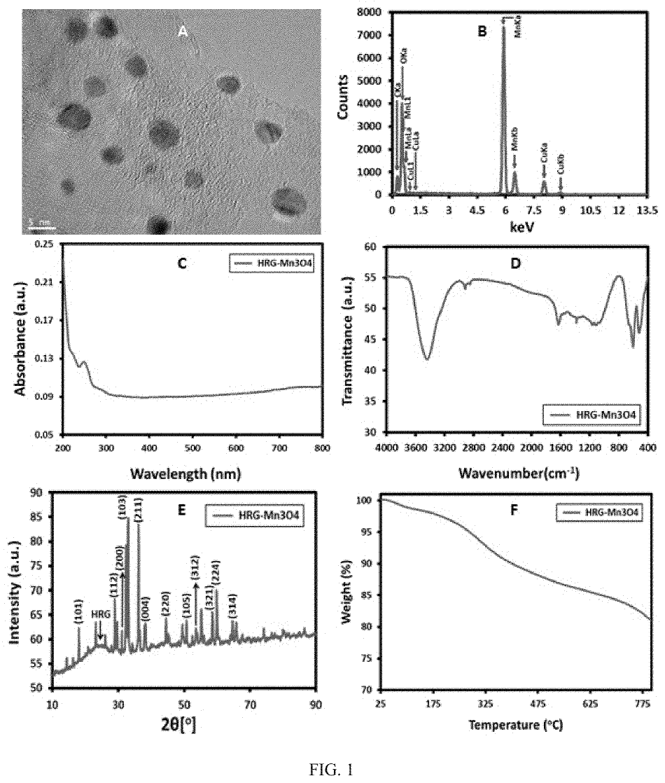

In one embodiment, the elements present in the hybrid nanoparticle (HRG-Mn₃O₄) e.g., as detected by an energy dispersive X-ray detector (EDX) are manganese, carbon, and oxygen. In more preferred embodiments, the nanocomposite is characterized by an energy-dispersive X-ray spectrum as shown in FIG. 1(B).

In an embodiment, the hybrid nanoparticle (HRG-Mn₃O₄) in accordance with the present disclosure is characterized by UV-visible spectrum comprising absorption bands at ˜220 and ˜270 nm respective to Mn₃O₄ and HRG for e.g., as shown in FIG. 1(C).

In an embodiment, the hybrid nanoparticle (HRG-Mn₃O₄) in accordance with the present disclosure is characterized by FT-IR spectrum comprising bands at ˜1630 cm⁻¹, ˜1209 cm⁻¹, ˜1050 cm⁻¹ and a broad band at ˜3440 cm⁻¹ respectively for C—O—C stretching, C—O stretching, C═C stretching, representing oxygen-containing functional groups selected from carbonyl, carboxylic, epoxy, and hydroxyl groups present in graphene oxide.

In an embodiment, the hybrid nanoparticle (HRG-Mn₃O₄) in accordance with the present disclosure is characterized by an FT-IR spectrum comprising absence of band at ˜1740 cm⁻¹, the relative decrease in the intensity of the bands at ˜3440 cm⁻¹ respectively representing the removal of oxygen-containing groups of graphene oxide in HRG, reduction of graphene oxide to HRG and presence of absorption bands at ˜624 cm⁻¹ and ˜525 cm⁻¹ respective to manganese representing the formation of HRG-Mn₃O₄ nanocomposite for e.g. as shown in FIG. 1(D).

In an embodiment, the hybrid nanoparticle (HRG-Mn₃O₄) in accordance with the present disclosure is characterized by the XRD pattern showing the characteristics peaks of both the entities (HRG as well as Mn₃O₄); the appearance of a broad peak at ˜22.4° (002) represent the reduction of graphene oxide for e.g., as shown in FIG. 1(E).

In an embodiment, the hybrid nanoparticle (HRG-Mn₃O₄) in accordance with the present disclosure is characterized by the weight loss of about 20% after heating up to 800° C. representing the presence of substantial oxygen functionalities for e.g., as shown in FIG. 1(F).

The hybrid nanoparticles (HRG-Mn₃O₄) in accordance with the present disclosure have excellent hemocompatibility with negligible RBC lysis.

The hybrid nanoparticles (HRG-Mn₃O₄) in accordance with the present disclosure do not pose any cytotoxicity at normal physiological conditions. Thus, the hybrid nanoparticles (HRG-Mn₃O₄) in accordance with the present disclosure are devoid of cytotoxicity at normal physiological conditions and are therefore biocompatible.

The hybrid nanoparticles (HRG-Mn₃O₄) in accordance with the present disclosure with exposure of laser light of specific wavelength resulted in massive cellular damage by HRG-Mn₃O₄ nanoparticles suggesting their potential for photodynamic therapy. The hybrid nanoparticles (HRG-Mn₃O₄) in accordance with the present disclosure also exhibit excellent MRI contrast property for tumor imaging.

In another embodiment, the present disclosure provides a method of synthesis of hybrid nanoparticle (HRG-Mn₃O₄) comprising steps of
(i). synthesising manganese oxide (Mn₃O₄) nanoparticles;
(ii). synthesising highly reduced graphene oxide (HRG) nanoparticles; and
(iii). preparing highly reduced graphene-Mn₃O₄ (HRG-Mn₃O₄) hybrid nanoparticles comprising steps of milling (Mn₃O₄) nanoparticles and highly reduced graphene oxide (HRG) nanoparticles.

In one embodiment, the synthesis of manganese oxide (Mn₃O₄) nanoparticles comprises steps of:
(i). dissolving manganese (II) acetylacetonate in oleylamine in a molar ratio of at least about 1:20 to about 1:30 to provide a slurry;
(ii). heating the slurry at least about 150° C. to about 170° C. for a period of at least about 8 hours to about 18 hours under a nitrogen atmosphere to provide a suspension;
(iii). separating a brown precipitate by centrifuging the suspension at least at about 7000 rpm to about 12000 rpm for at least about 5 mins to about 30 mins; and
(iv). washing the precipitate with a C1-C3 alcohol multiple times to obtain manganese oxide (Mn₃O₄) nanoparticles.

In one embodiment, the molar ratio of manganese (II) acetylacetonate to oleylamine can be selected from at least about 1:20, 1:21, 1:22, 1:23, 1:24, 1:25, 1:26, 1:27, 1:28, 1:29 and 1:30.

In one embodiment, the temperature for heating the slurry obtained in step (ii) can be at least at about 150° C., 155° C., 160° C., 165° C., 170° C., or any temperature in between said temperatures.

In one embodiment, the period for heating the slurry obtained in step (ii) can be at least about 8 hours, 9 hours, 10 hours, 11 hours, 12 hours, 13 hours, 14 hours, 15 hours, 16 hours, 17 hours or 18 hours.

In one embodiment, the separating of the brown precipitate by centrifuging the suspension can be carried out at speed of at least about 7000 rpm, 8000 rpm, 9000 rpm, 10000 rpm, 11000 rpm or 12000 rpm.

In a preferred embodiment, the C1-C3 alcohol used for washing the precipitate is ethyl alcohol.

In one embodiment, the synthesised manganese oxide (Mn₃O₄) nanoparticles can be re-dispersed in organic solvent selected from but not limiting to hexane, toluene, and dichloromethane.

In one embodiment, the synthesised manganese oxide (Mn₃O₄) nanoparticles can be dried in a vacuum before use.

In one embodiment, the synthesis of highly reduced graphene oxide (HRG) nanoparticles comprises steps of:
(i). synthesizing a graphene oxide (GRO) from graphite powder;
(ii). converting the graphene oxide (GRO) to a highly reduced graphene oxide (HRG) comprising steps:
   a) dispersing GRO in water and sonicating for at least about 10 mins to about 60 mins to provide a suspension;
   b) heating the suspension up to 100° C. and adding about 1 ml to about 5 ml of hydrazine hydrate and continuing the reaction under reduced temperature of at least about 95° C. to about 98° C. under stirring for a period of about 18 hours to about 28 hours to provide a suspension;
   c) centrifuging the suspension at least at about 2000 rpm to about 5000 rpm for about 2 mins to about 10 mins to obtain a filtrate;
   d) washing the filtrate several times with water and drying under vacuum to provide a black powder of HRG.

In one embodiment, sonication of GRO dispersed in water in step (iii)(a) can be carried out at least for about 10 mins, 15 mins, 20 mins, 25 mins, 30 mins, 35 mins, 40 mins, 45 mins, 50 mins, 55 mins, or about 60 mins to provide a suspension.

In one embodiment, in step (iii)(b), the amount of hydrazine hydrate is added is about 1 ml, 2 ml, 3 ml, 4 ml, or 5 ml.

In one embodiment, in step (iii)(b), the reduced temperature for carrying out the reaction upon addition of hydrazine hydrate is selected from about 95° C., 96° C., 97° C., or 98° C.

In one embodiment, in step (iii)(b), the reaction after adding hydrazine hydrate is continued for a period selected from of about 18 hours, 19 hours, 20 hours, 21 hours, 22 hours, 23 hours, 24 hours, 25 hours, 26 hours, 27 hours, or 28 hours to provide a suspension.

In one embodiment, in step (iii)(c), the centrifugation of the suspension is carried out for at least at about 2000 rpm, 3000 rpm, 4000 rpm, or 5000 rpm.

In one embodiment, in step (iii)(c), the centrifugation of the suspension is carried out at least for about 2 mins, 3, mins, 4 mins, 5 mis, 6 mins, 7 mins, 8 mins, 9 mis and 10 mins.

In one embodiment, the preparing (HRG-Mn₃O₄) hybrid nanoparticles comprises steps of:
(i). milling manganese oxide (Mn₃O₄) nanoparticles and highly reduced graphene oxide (HRG) nanoparticles in a ratio of about 1:0.5 to about 1:2, preferably at a ratio of about 1:1; and
(ii). continuing milling for a period of at least about 12 hours to 20 hours with intermittent pause(s).

In one embodiment, the milling of manganese oxide (Mn₃O₄) nanoparticles and highly reduced graphene oxide (HRG) nanoparticles is continued for a period selected from about 12 hours, 13 hours, 14 hours, 15 hours, 16 hours, 17 hours, 18 hours, 19 hours or 20 hours.

In an embodiment, the milling of manganese oxide (Mn₃O₄) nanoparticles and highly reduced graphene oxide (HRG) nanoparticles is carried out in the presence of stainless steel balls of about 5 mm diameter with the ball to powder weight ratio of about 1:1 to provide highly reduced graphene-Mn₃O₄ (HRG-Mn₃O₄) hybrid nanoparticles.

The highly reduced graphene-Mn₃O₄ (HRG-Mn₃O₄) hybrid nanoparticles in accordance with the present disclosure are useful for theranostic applications for example diagnosis and treatment of cancer. The highly reduced graphene-Mn₃O₄ (HRG-Mn₃O₄) hybrid nanoparticles of the present disclosure are useful for optical and MRI imaging technique for both in-vivo animal and clinical cancer diagnosis. The highly reduced graphene-Mn₃O₄ (HRG-Mn₃O₄) hybrid nanoparticles in accordance with the present disclosure are useful for photodynamic therapy.

In another embodiment, the present disclosure provides a use of (HRG-Mn₃O₄) hybrid nanoparticles for photodynamic therapy of cancer.

In another embodiment, the present disclosure provides a use of (HRG-Mn₃O₄) hybrid nanoparticles as MRI imaging agent or contrast agent to enhance the magnetic resonance signals from cancer cells.

In another embodiment, the present disclosure provides use of (HRG-Mn₃O₄) hybrid nanoparticles as photodynamic therapy agent for treatment of cancer and/or as contrast agent for imaging of cancer cells.

Other objects, advantages and features of the present invention will become more apparent upon reading of the following non-restrictive description of specific embodiments thereof, given by way of example only with reference to the accompanying drawings.

### EXAMPLES

The present invention is illustrated in further details by the following non-limiting examples.

### Example 1

Preparation of (HRG-Mn₃O₄) Hybrid Nanoparticles

### Chemicals and Reagents:

All chemicals including solvents used for the synthesis of nanoparticles were procured from Sigma Aldrich (St. Louis, Mo., USA). Graphite powder (99.999%, 200 mesh) was purchased from Alfa Aesar, Kandel, Germany. Deionized water was prepared from a Millipore Milli-Q system and used in all experiments.

A) Synthesis of Mn₃O₄ Nanoparticles:

A slurry of Manganese (II) acetylacetonate was dissolved in oleylamine (molar ratio of Manganese (II) acetylacetonate:oleylamine=1:25) in a 100 mL three-neck flask. The mixture was heated at 162° C. for 11 hrs. under a nitrogen atmosphere, and then the resulting mixture was cooled down to room temperature to form a brown suspension. After centrifugation at 9000 rpm for 15 mins, the supernatant was removed and a brown precipitate was obtained. The brown precipitate was washed with ethanol five times to acquire pure Mn₃O₄ nanoparticles. Finally, Mn₃O₄ nanoparticles were dried in a vacuum before use.

### B) Synthesis of Highly Reduced Graphene Oxide (HRG):

Initially graphite oxide (GO) was synthesized from graphite powder and then using a modified Hummers method (Hummers W S, Offeman R E., Preparation of graphitic oxide, J Am Chem Soc. 1958; 80: 1339; and Alam S N, Sharma N, Kumar L., Synthesis of graphene oxide (GRO) by modified Hummers method and its thermal reduction to obtain highly reduced graphene oxide (HRG), Graphenes 2017; 6: 1-18) and then it was converted to highly reduced graphene oxide (HRG) following several steps of centrifugation, washing and finally sonication. GRO was reduced according to a previously reported method (Assal M E, Shaik M R, Kuniyil M, Khan M, Alzahrani A Y, Al-Warthan A, Siddiqui M R H, Adil S F., Mixed zinc/manganese on highly reduced graphene oxide: A highly active nanocomposite catalyst for aerial oxidation of benzylic alcohols, Catalysts 2017; 7: 391). Briefly, GRO was dispersed in water and sonicated for 30 min. The resulting suspension was allowed to heat up to 100° C. and subsequently 3 ml of hydrazine hydrated were added. The temperature was slightly reduced (98° C.), and the suspension was kept under stirring for 24 h. Finally, a black powder was obtained which was filtered and washed several times with water. The resultant suspension was centrifuged at 4,000 rpm for several 3 min, and the final product was collected via filtration and dried under vacuum.

C) Preparation of Highly Reduced Graphene-Mn₃O₄ (HRG-Mn₃O₄) Hybrid Nanoparticles:

Approximately 200 mg of Mn₃O₄ nanoparticles and 200 mg of highly reduced graphene (HRG) powder were milled using Fritsch Pulverisette P7 (Idar-Oberstein, Germany) planetary ball mill. The nanocomposite powder and stainless steel balls (5 mm diameter) with the ball to powder weight ratio of 1:1 were introduced into the stainless steel container. The milling of the powder was performed for 16 hours. The milling process was paused at regular intervals to give (HRG-Mn₃O₄) hybrid nanoparticles.

### Example 2

Characterization of (HRG-Mn₃O₄) Hybrid Nanoparticles

The synthesized (HRG-Mn₃O₄) hybrid nanoparticles were characterized for size, elemental composition, physicochemical properties and stability using high resolution transmission electron microscopy (JEM-2100F, JEOL, Japan), energy-dispersive X-ray spectroscopy (EDX), UV-Vis spectroscopy (Perkin Elmer lambda 35, Waltham, Mass., USA), FT-IR spectroscopy (Perkin Elmer 1,000 FT-IR spectrometer), X-ray diffraction analysis (D2 Phaser X-ray diffractometer, Bruker, Germany) and thermogravimetric analysis (TGA/DSC1, Mettler Toledo AG, Analytical, Schwerzenbach, Switzerland).

The shape of hybrid nanoparticles appeared as round with the average diameter of 12±2.21 nm (FIG. 1(A)).

In EDX analysis, the intense signals at 0.65, 5.88, and 6.62 keV strongly suggests that ‘Mn’ was the major element, which has an optical absorption in this range owing to the surface plasmon resonance (SPR). The other signals found in the range 0.0-0.5 keV signify the absorption of carbon and oxygen, confirming the formation of HRG-Mn₃O₄ nanocomposite (FIG. 1(B)).

UV-visible spectrum of HRG-Mn₃O₄ nanoparticles showed respective absorption bands at ˜220 (Mn₃O₄) and ˜270 nm (HRG) indicating the formation of HRG-Mn₃O₄ (FIG. 1(C)).

FT-IR spectrum of HRG-Mn₃O₄ displayed the graphene oxide bands at ˜1630 cm⁻¹ (for C═C stretching), ˜1209 cm⁻¹ (for C—O—C stretching), ˜1050 cm⁻¹ (for C—O stretching), and a broad band at around 3440 cm⁻¹ for hydroxyl groups indicated the presence of various oxygen-containing functional groups, such as carbonyl, carboxylic, epoxy, and hydroxyl groups in graphene oxide. The removal of oxygen-containing groups of graphene oxide in HRG was indicated by the disappearance of some of the bands such as the band at ˜1740 (which is present in HRG only; spectrum not shown). Also, the relative decrease in the intensity of some of the bands, like the decrease in intensity of broad band at 3440 cm⁻¹ points towards the reduction of graphene oxide. The existence of other absorption bands of Mn at 624 and 525 cm⁻¹ clearly indicated the formation of HRG-Mn₃O₄ nanocomposite (FIG. 1(D)).

The XRD pattern of HRG-Mn₃O₄ nanoparticles showed the characteristics peaks of both the entities (HRG as well as Mn₃O₄); the appearance of a broad peak at ˜22.4° (002) confirmed the reduction of graphene oxide (FIG. 1(E)).

TGA analysis of HRG-Mn₃O₄ nanoparticles displays the weight loss of about 20% after heating up to 800° C. indicating the presence of substantial oxygen functionalities (FIG. 1(F)).

### Example 3

Biological Assays of (HRG-Mn₃O₄) Hybrid Nanoparticles

### Statistics:

All the cell based analyses were performed in triplicate and the results reported as means±standard deviation. The data were analyzed by one-way analysis of variance (ANOVA) followed by Dunnett's test. P values<0.05 were considered as statistically significant.

### I. Hemolysis Assay:

Hemolysis assay was performed by collecting 2 mL of blood from Sprague Dawley rat (6 weeks, Female) through cardiac puncture. The blood drawn from the rat was immediately centrifuged at 1500 rpm for 10 min. The pellet was washed with PBS three times and finally suspended in 20 mL of PBS. Then, 500 μL of freshly prepared RBCs were added to different concentrations (12, 25, 50 and 100 μg mL-1) of nanoparticles (500 μL), as well as positive (PBS) and negative control (H₂O) tubes. All of samples were prepared in triplicate. The samples were then placed in a 37° C. incubator for 4 hours and then centrifuged at 1500 rpm for 10 min. The absorbance of the supernatant was recorded at 540 nm and collected the digital image. Hemolysis percent was calculated by diving each sample's hemoglobin concentration by the total blood hemoglobin as per the following equation:

Hemolysis %=(Hemoglobin in test sample/Total Blood Hemoglobin)×100.

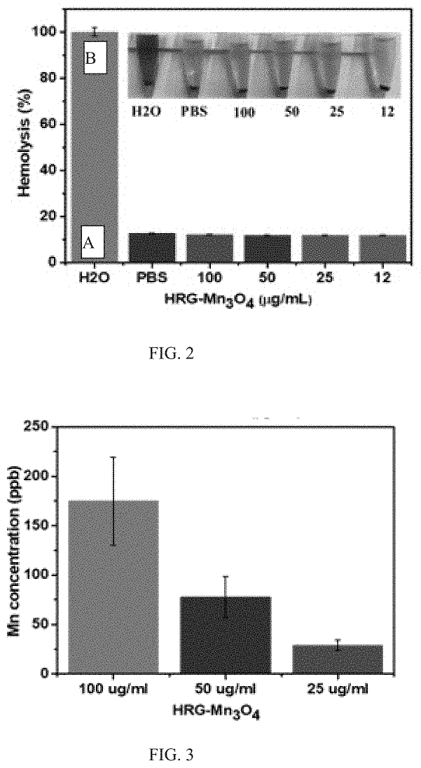

No obvious hemolysis was observed when the RBCs were incubated even with a higher concentration (100 μg mL⁻¹) of nanoparticles. In addition, the extent of lysis was similar to PBS implied that developed HRG-Mn₃O₄ nanoparticles had excellent hemocompatibility with negligible RBC lysis (FIG. 2). This result confirmed that the HRG-Mn₃O₄ nanoparticles are biocompatible.

### II. Cytotoxicity Assay:

In-vitro cytotoxicity assay to investigate the toxicity profile of newly synthesized HRG-Mn₃O₄ hybrid nanoparticles was performed. In vitro cytotoxicity studies of nanoparticles are preferred as they are simple, cost-effective and faster than in-vivo models.

The cytotoxicity of Mn₃O₄ and HRG-Mn₃O₄ nanoparticles was performed by 3-(4,5-dimethylthiazol-2-yl)-2,5-diphenyltetrazolium bromide (MTT) method. A549 cells (a non-small cell lung cancer cell line comprising lung carcinoma epithelial cells) were seeded in the 96-well plate (4×10⁴ cells per well) in RPMI medium and incubated in the atmosphere of 5% CO₂ at 37° C. for 24 hrs. Different concentrations (6.25, 12.5, 25, 50 and 100 μg/ml) of Mn₃O₄ and HRG-Mn₃O₄ were added to the respective wells of micro plate.

For laser-induced phototoxicity analysis, the nanoparticles and further incubated for 4 h. Phosphate buffer saline (PBS) and triton X-100 were used as control and negative control, respectively. Then cells were treated with a 670 nm laser irradiation at 0.1 W/cm² for 5 min and further incubated for 24 h. MTT aqueous solution (50 ml) was added to each well in the 96-well plate 4 h before the termination of 24 h incubation. The upper layer of the solution was discarded. The MTT solubilization solution, DMSO (100 μL) was added to each well to dissolve the formazan crystals by pipette stirring and then observed absorbance at 590 nm, which was converted to cell viability based on absorbance of dissolved formazan. The cellular uptake was performed by ICP-MS. The viable quantity of cells was then calculated using the following equation:

Cell viability (%)=(absorbance of sample cells/absorbance of control cells)×100

The results showed that developed nanoparticles are capable of cellular interactions to A549 cells at different concentrations (100, 50, 25 μg mL⁻¹) while the cellular uptake was directly proportional with the concentration of nanoparticles (FIG. 3).

The cytotoxicity analysis using MTT assay showed that more than 98% of A549 cells survived even after the exposure of a high concentration (100 μg/ml) of nanomaterials indicating the biocompatibility of both Mn₃O₄ and HRG-Mn₃O₄ nanoparticles (FIG. 4).

These results confirmed that the HRG-Mn₃O₄ nanoparticles can easily be taken up by cells (FIG. 3) and are nontoxic (FIG. 4) under physiological conditions.

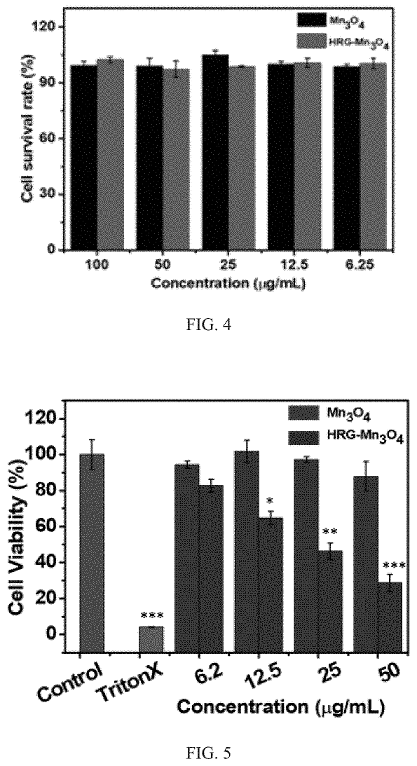

Almost 100% cells were viable when treated with phosphate buffered saline (PBS) or Mn₃O₄ nanoparticles in presence of 670 nm wavelength laser irradiation (0.1 W/cm²) for 5 min (FIG. 5). However, laser irradiation resulted in significant and concentration-dependent cellular damage by HRG-Mn₃O₄ nanoparticles (FIG. 5). These results confirm a significant and concentration-dependent cytotoxicity of HRG-Mn₃O₄ after laser irradiation as compared to Mn₃O₄ nanoparticles.

### III. In-Vitro Photodynamic Therapy Using Fluorescence Microscopy of Live and Dead Cells:

The live/dead assay kit containing fluorescein diacetate (FDA) and propidium iodide (PI) to visualize live and dead cells, respectively was used and cells were visualized under fluorescence microscope. A549 cells (2×10⁴ cells per well) were seeded on a 24 well plate and incubated in the atmosphere of 5% CO₂ at 37° C. for 24 hrs. Mn₃O₄ and HRG-Mn₃O₄ nanoparticles (50 μg/ml) were added to the 24 well plate. PBS was used as a control and the plate was incubated for 4 h. Then cells were exposed to a 670 nm wavelength laser irradiation at 0.1 W/cm² for 5 min and further incubated for 24 h. FDA and PI were added to treated cells and incubated for 5 min. Then cells were washed with PBS thrice to remove excess FDA/PI and fluorescence images were acquired by fluorescence microscope with 490 nm excitation and emission at 525 nm.

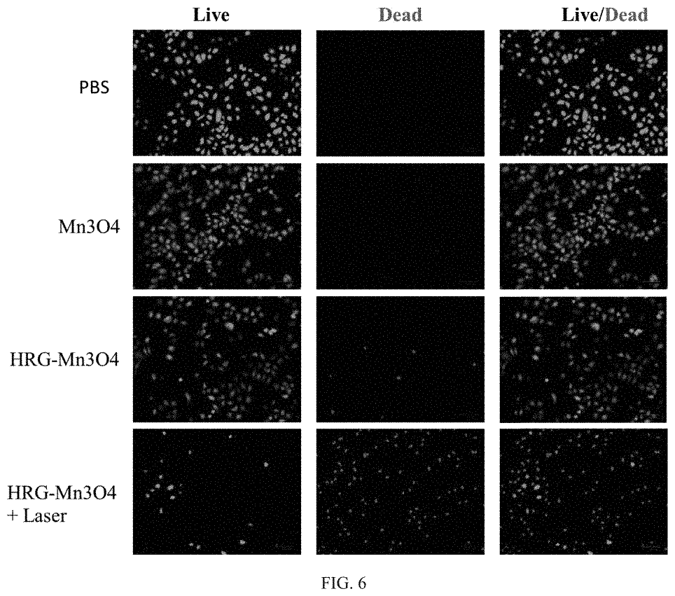

To study the interactions between cells and the nanoparticles, we used the visible red optical imaging of A549 cells after incubation in PBS, Mn₃O₄ nanoparticles, HRG-Mn₃O₄ nanoparticles and HRG-Mn₃O₄ nanoparticles with and without laser irradiation for 5 min. (FIG. 6). After 5 min, propidium iodide (PI) (red emission for dead cells) fluorescent dots were observed in HRG-Mn₃O₄ nanoparticles plus laser treated group when compared to HRG-Mn₃O₄ nanoparticles treated cells. However, no red fluorescent dots were observed in PBS and Mn₃O₄ treated A549 groups of cells. On the other hand, A549 cells incubated in PBS, Mn₃O₄ nanoparticles, HRG-Mn₃O₄ nanoparticles showed abundant green emission indicating the presence of live cells (FIG. 6). This result confirm that hybrid nanoparticles produced cytotoxicity only after laser irradiation suggesting their potential for PDT of cancer.

### IV. In-Vitro MRI Imaging:

A series of aqueous solutions of HRG-Mn₃O₄ nanoparticles (with Mn from 0 to 1 mM) were prepared and imaged in a 0.2 Eppendorf tube on a 3 T MRI instrument. A series of cell culture medium of HRG-Mn₃O₄ nanoparticles (0 to 50 mg/mL) were treated with A549 cells (1*104) per well. After 4 h incubation, cells were washed with PBS and collected for MRI imaging using a 3 T MRI scanner (BioSpec 47/40; Bruker, Germany) at Korea Basic Science Institute with 72 mm and 35 mm volume coils for phantom.

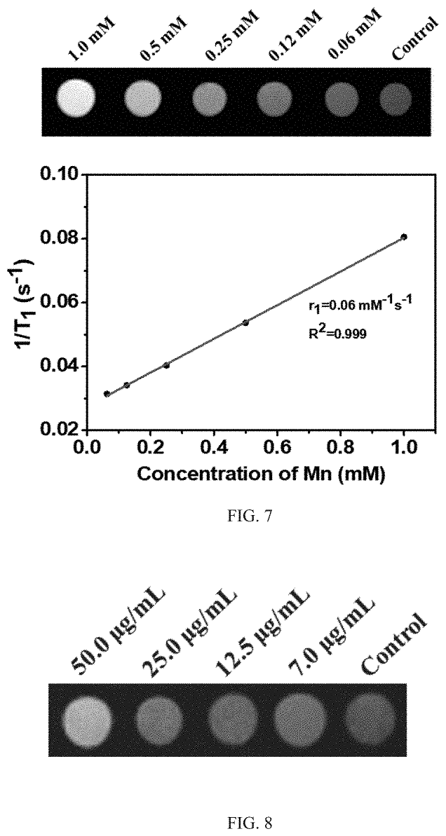

The results of MRI analysis demonstrated a significant enhancement of signal intensity of nanoparticles with increasing Mn concentration using nanoparticles suspension (FIG. 7) as well as cells exposed to nanoparticles (FIG. 8). The specific relaxivity (r1) was calculated from linear curve generated from concentration of Mn versus 1/T₁ (s⁻¹). The r1 value was found to be 0.06 mM⁻¹s⁻¹. The results of MRI showed concentration dependent contrast property of HRG-Mn₃O₄ nanoparticles in both aqueous suspension (FIG. 7) and cancer cells (FIG. 8). Thus, they have higher T1 or T2 relaxivity and meet the requirements for clinical usefulness.

These results confirmed that the HRG-Mn₃O₄ nanoparticles do not pose any cytotoxicity at normal physiological conditions and therefore they are biocompatible. However, exposure of laser light of specific wavelength resulted in massive cellular damage by HRG-Mn₃O₄ nanoparticles suggesting their potential for photodynamic therapy. The newly synthesized nanoparticles also showed excellent MRI contrast property for tumor imaging. Thus, these results show capability of the HRG-Mn₃O₄ nanoparticles for theranostic applications.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as a limitation.

What is claimed is:

1. Hybrid nanoparticles (HRG-Mn₃O₄) as photodynamic therapy agent and imaging agent for cancer.

2. The hybrid nanoparticles of claim 1, wherein the hybrid nanoparticles (HRG-Mn₃O₄) are round with the average diameter of about 8 nm to about 16 nm, preferably from about 9 nm to about 15 nm.

3. The hybrid nanoparticles of claim 1, wherein the hybrid nanoparticle (HRG-Mn₃O₄) exhibits X-ray photoelectron spectrum comprising the peaks at about 0.65 keV, 5.88 keV, and 6.62 keV, and 0.0-0.5 keV.

4. The hybrid nanoparticles of claim 1, wherein the elements present in the hybrid nanoparticle (HRG-Mn₃O₄) e.g., as detected by an energy dispersive X-ray detector (EDX) are manganese, carbon, oxygen, and. In more preferred embodiments, the nanocomposite is characterized by an energy-dispersive X-ray spectrum as shown in FIG. 1(B).

5. The hybrid nanoparticles of claim 1, wherein the hybrid nanoparticle (HRG-Mn₃O₄) is characterized by UV-visible spectrum comprising absorption bands at ˜220 and ˜270 nm respective to Mn₃O₄ and HRG.

6. The hybrid nanoparticles of claim 1, wherein the hybrid nanoparticle (HRG-Mn₃O₄) is characterized by FT-IR spectrum comprising bands at ˜1630 cm⁻¹, ˜1209 cm⁻¹, ˜1050 cm⁻¹ and a broad band at ˜3440 cm⁻¹.

7. The hybrid nanoparticles of claim 1, wherein the hybrid nanoparticle (HRG-Mn₃O₄) is characterized by an FT-IR spectrum comprising absence of band at ˜1740 cm⁻¹, the decrease in the intensity of the bands at ˜3440 cm⁻¹ and presence of absorption bands at ˜624 cm⁻¹ and ˜525 cm⁻¹.

8. The hybrid nanoparticles of claim 1, wherein hybrid nanoparticle (HRG-Mn₃O₄) is characterized by The XRD pattern showing a broad peak at ˜22.4° (002) confirming the reduction of graphene oxide.

9. The hybrid nanoparticles of claim 1, wherein the hybrid nanoparticle (HRG-Mn₃O₄) is characterized by the weight loss of about 20% after heating up to 800° C.

10. The hybrid nanoparticles of claim 1, wherein the hybrid nanoparticles (HRG-Mn₃O₄) are hemocompatible.

11. The hybrid nanoparticles of claim 1, wherein the hybrid nanoparticles (HRG-Mn₃O₄) do not cause cytotoxicity at normal physiological conditions.

12. The hybrid nanoparticles of claim 1, wherein the hybrid nanoparticles (HRG-Mn₃O₄) cause cellular damage to cancer cells upon exposure of laser light of 670 nm wavelength at a light intensity of 4 mW cm².

13. A method of synthesis of hybrid nanoparticle (HRG-Mn₃O₄) comprising steps of:
(i). synthesising manganese oxide (Mn₃O₄) nanoparticles;
(ii). synthesising highly reduced graphene oxide (HRG) nanoparticles; and
(iii). preparing highly reduced graphene-Mn₃O₄ (HRG-Mn₃O₄) hybrid nanoparticles comprising steps of milling (Mn₃O₄) nanoparticles and highly reduced graphene oxide (HRG) nanoparticles.

14. The method of synthesis of hybrid nanoparticle of claim 13, wherein the synthesis of manganese oxide (Mn₃O₄) nanoparticles comprises steps of:
(i). dissolving manganese (II) acetylacetonate in oleylamine in a molar ratio of 1:20 to 1:30 to provide a slurry;
(ii). heating the slurry at about 150° C. to 170° C. for a period of about 8 hours to about 18 hours under a nitrogen atmosphere to provide a suspension;
(iii). separating a brown precipitate by centrifuging the suspension at about 7000 rpm to about 12000 rpm for about 5 mins to about 30 mins; and
(iv). washing the precipitate with a C1-C3 alcohol multiple times to obtain manganese oxide (Mn₃O₄) nanoparticles.

15. The method of synthesis of hybrid nanoparticle of claim 13, wherein the synthesis of highly reduced graphene oxide (HRG) nanoparticles comprises steps of:
(i). synthesizing a graphene oxide (GRO) from graphite powder;
(ii). converting the graphene oxide (GRO) to a highly reduced graphene oxide (HRG) comprising steps:
a) dispersing GRO in water and sonicating for about 10 mins to about 60 mins to provide a suspension;
b) heating the suspension up to 100° C. and adding about 1 ml to about 5 ml of hydrazine hydrate and continuing the reaction under reduced temperature of about 95° C. to about 98° C. under stirring for a period of about 18 hours to about 28 hours to provide a suspension;
c) centrifuging the suspension at about 2000 rpm to about 5000 rpm for about 2 mins to about 10 mins to obtain a filtrate;
d) washing the filtrate several times with water and drying under vacuum to provide a black powder of HRG.

16. The method of synthesis of hybrid nanoparticle of claim 13, wherein the preparing (HRG-(Mn₃O₄)) hybrid nanoparticles comprises steps of:
(i). milling manganese oxide (Mn₃O₄) nanoparticles and highly reduced graphene oxide (HRG) nanoparticles in a ratio of about 1:0.5 to about 1:2, preferably at a ratio of about 1:1; and
(ii). continuing milling for a period of about 12 hours to 20 hours with intermittent pause(s).

17. Use of (HRG-Mn₃O₄) hybrid nanoparticles as photodynamic therapy agent for treatment of cancer and/or as contrast agent for imaging of cancer cells.
